(19) United States
(12) Patent Application Publication
(10) Pub. No.: US 2023/0119530 A1
(43) Pub. Date: Apr. 20, 2023

(54) GAS SENSOR

(71) Applicant: NGK SPARK PLUG CO., LTD., Nagoya-shi, Aichi (JP)

(72) Inventors: Kunihiko YONEZU, Nagoya-shi, Aichi (JP); Yuji SHIMAZAKI, Nagoya-shi, Aichi (JP); Masayuki YOSHIDA, Nagoya-shi, Aichi (JP)

(73) Assignee: NGK SPARK PLUG CO., LTD., Nagoya-shi, Aichi (JP)

(21) Appl. No.: 17/905,219

(22) PCT Filed: Apr. 22, 2021
(86) PCT No.: PCT/JP2021/016261

§ 371 Date: Aug. 29, 2022

(30) Foreign Application Priority Data
Oct. 29, 2020 (JP) 2020-181914

(51) Int. Cl. G01N 27/407; G01N 33/00

(52) U.S. Cl. CPC G01N 27/4077; G01N 33/0027

(57) ABSTRACT

A gas sensor (1) including: a sensor element (21) having a detection section (22) through an element introduction hole (25); a metallic shell (11); and a single-wall tubular protector (51) having a gas introduction hole (56) and a gas discharge hole (53); a gap G is present between the gas introduction hole and a forwardly facing surface (12a) of the metallic shell; when the gas introduction hole is viewed toward the rear end side, an area Sh of a portion of the forwardly facing surface seen through the gas introduction hole, is equal to or greater than ½ of an opening area Sg of the gas introduction hole; the element introduction hole is located on the forward end side in relation to a forwardmost end (12f) of the forwardly facing surface; and a distance L1 of the gap G is smaller than a diameter D of the gas introduction hole.

### CROSS-REFERENCE TO RELATED APPLICATIONS

The application is a National Stage of International Application No. PCT/JP2021/016261 filed Apr. 22, 2021, which claims Japanese Patent Application No. 2020-181914, filed Oct. 29, 2020, incorporated herein by reference in its entirety.

### BACKGROUND OF THE INVENTION

### 1. Field of the Invention

The present invention relates to a gas sensor having a single-wall protector.

### 2. Description of the Related Art

Conventionally, a gas sensor has been known in which a sensor element is held by a tubular metallic shell, and a forward end portion of the sensor element, which portion is exposed to exhaust gas, is protected by a single-wall or double-wall protector. A gas introduction hole is provided in this protector. The gas introduction hole secures wetting resistance by preventing condensed water mixed in the exhaust gas from reaching the sensor element, and secures responsiveness by quickly introducing the exhaust gas into a detection section of the sensor element. The sensor element is heated by a heater of the sensor element or by exhaust gas of high temperature. Therefore, if condensed water comes into contact with the sensor element, the sensor element may break due to thermal shock.

In view of the above, a technique has been developed of improving responsiveness by employing a single-wall protector and providing a gas introduction hole in a horizontal step portion of the protector so that the gas introduction hole is located on the forward end side of the metallic shell (Patent Document 1). According to this technique, the exhaust gas first flows from the gas introduction hole toward the metallic shell, and then changes its flow direction in an internal space between the gas introduction hole and the metallic shell so that the exhaust gas flows toward the forward end side within the protector. Therefore, due to the weight of the condensed water itself, the condensed water is more likely to separate from the exhaust gas.
Patent Document 1: Japanese Patent Application Laid-Open (kokai) No. 2019-70601 (FIG. 1)

### 3. Problems to be Solved by the Invention

However, if the condensed water flowing from the metallic shell side after having its flow direction changed reaches the detection section of the sensor element, element breakage or a like problem may occur, resulting in an insufficient degree of wetting resistance.

### SUMMARY OF THE INVENTION

The present invention has been accomplished to solve the above-noted problems, and an object thereof is to provide a gas sensor which can enhance both wetting resistance and responsiveness by using a single-wall protector.

The above object of the present invention has been achieved, in a first object, by providing (1) a gas sensor comprising: a sensor element which extends in an axial direction and has a detection section formed in a forward end portion and detecting a gas to be detected through an element introduction hole; a tubular metallic shell which surrounds a circumference of the sensor element in a radial direction and holds the sensor element; and a single-wall tubular protector fixed to a circumference of a forward end portion of the metallic shell and surrounding the forward end portion of the sensor element. The protector has a gas introduction hole facing a rear end side and a gas discharge hole disposed on a forward end side in relation to the gas introduction hole. A gap G in the axial direction is present between the gas introduction hole and a forwardly facing surface of the metallic shell. Further, when the gas introduction hole is viewed toward the rear end side in the axial direction, an area Sh of a portion of the forwardly facing surface of the metallic shell, which portion can be seen through the gas introduction hole, is equal to or greater than ½ of an opening area Sg of the gas introduction hole. The entirety of the element introduction hole is located on the forward end side in relation to a forwardmost end of the forwardly facing surface of the metallic shell; and a distance L1 of the gap G is smaller than a diameter D of the gas introduction hole.

In the above gas sensor (1), the distance L1 is rendered smaller than the diameter D. Therefore, when a water droplet flows, together with the gas to be detected, from the gas introduction hole toward the metallic shell, the water droplet strikes the forwardly facing surface of the metallic shell without fail. Consequently, the water droplet is broken to form fine water droplets. Therefore, even when the water droplets change their flow direction, flow toward the forward end side within the protector, and come into contact with the forward end portion of the sensor element, the sensor element receives a reduced thermal shock as compared to the case where a water droplet having a large diameter comes into contact with the sensor element. Therefore, the sensor element is less likely to break and its wetting resistance can be enhanced.

Since the relation of Sh≥½×Sg is satisfied, one half or more of the opening area of the gas introduction hole faces the forwardly facing surface of the metallic shell. Therefore, the possibility of the water droplet flowing from the gas introduction hole toward the metallic shell to strike the forwardly facing surface is increased. As a result, such configuration can more reliably cause the water droplet to strike the forwardly facing surface, and thereby form fine water droplets.

Further, since the protector is single walled, the gas to be detected flows easily into the protector as compared with a double-wall protector, and responsiveness is improved.

Notably, the reason why the distance L1 is rendered smaller than the diameter D is as follows. The maximum diameter of water droplets introduced into the protector through the gas introduction hole is D. Therefore, if the distance L1 is equal to or larger than the diameter D, when a water droplet having a diameter D enters the gap G through the gas introduction hole, there is a possibility that the water droplet moves within the protector without striking the forwardly facing surface and comes into contact with the sensor element while maintaining its large size.

In a second aspect, the present invention provides (2) a gas sensor comprising: a sensor element which extends in an axial direction and has a detection section formed in a forward end portion and detecting a gas to be detected through an element introduction hole; a tubular metallic shell which surrounds a circumference of the sensor element in a radial direction and holds the sensor element; and a single-wall tubular protector fixed to a circumference of a forward end portion of the metallic shell and surrounding the forward end portion of the sensor element. The protector has a gas introduction hole facing a rear end side and a gas discharge hole disposed on a forward end side in relation to the gas introduction hole. A gap G in the axial direction is present between the gas introduction hole and a forwardly facing surface of the metallic shell. Further, when the gas introduction hole is viewed toward the rear end side in the axial direction, an area Sh of a portion of the forwardly facing surface of the metallic shell, which portion can be seen through the gas introduction hole, is equal to or greater than ½ of an opening area Sg of the gas introduction hole. The entirety of the element introduction hole is located on the forward end side in relation to a forwardmost end of the forwardly facing surface of the metallic shell; and a distance L1 of the gap G is smaller than a distance L2 between the sensor element and the gas introduction hole in the radial direction.

In this gas sensor, the distance L1 is rendered smaller than the distance L2. Therefore, when a water droplet flows, together with the gas to be detected, from the gas introduction hole toward the metallic shell, the water droplet strikes the forwardly facing surface of the metallic shell without fail, whereby the water droplet is broken to form fine water droplets. Therefore, even when the water droplets change their flow direction, flow toward the forward end side within the protector, and come into contact with the forward end portion of the sensor element, the sensor element receives a reduced thermal shock as compared to the case where a water droplet having a large diameter comes into contact with the sensor element. Therefore, the sensor element becomes less likely to break and its wetting resistance can be enhanced.

Since the relation of Sh≥½×Sg is satisfied, one half or more of the area of the forwardly facing surface of the metallic shell faces the gas introduction hole. Therefore, the possibility of the water droplet flowing from the gas introduction hole toward the metallic shell to strike the forwardly facing surface increases, whereby such configuration can more reliably cause the water droplet to strike the forwardly facing surface, and thereby form fine water droplets.

Further, since the protector is single walled, the gas to be detected flows easily into the protector as compared with a double-wall protector, and responsiveness is improved.

Notably, the diameter (the maximum diameter) of a largest water droplet which enters the gap G through the gas introduction hole and then moves within the protector without striking the forwardly facing surface is equal to the distance L1. In view of the above, the distance L1 is rendered smaller than the distance L2. In this case, even when a water droplet has a diameter equal to the distance L1 (the maximum diameter), the water droplet flows while keeping distance from the sensor element. Therefore, the possibility of the water droplet coming into contact with the sensor element decreases.

In a preferred embodiment (3) of any of the above gas sensors (1) and (2), the forwardly facing surface of the metallic shell constitutes a horizontal surface parallel to the radial direction and/or a taper surface which tapers toward the forward end side and faces a radially outer side.

In the gas sensor (3), a water droplet having flowed through the gas introduction hole to strike the horizontal surface or the taper surface flows downward (the horizontal surface) or flows toward the radially outer side (the taper surface). Namely, the water droplet bounces in directions which are not directions toward the radially inner side. Therefore, the water droplet becomes less likely to come into contact with the sensor element.

In contrast, in the case where the forwardly facing surface of the metallic shell has a “taper surface which tapers toward the forward end side and faces toward the radially inner side,” a water droplet striking this taper surface bounces toward the radially inner side and approaches the sensor element. Therefore, the water droplet becomes more likely to come into contact with the sensor element, whereby the sensor element is wetted with water. As a result, wetting resistance may decrease.

In another preferred embodiment (4) of any of the above gas sensors (1), (2) and (3), when the gas introduction hole is viewed from an outer side in the radial direction, the interior of the protector cannot be seen.

The gas sensor (4) can prevent a water droplet from entering the protector from a radial direction, thereby preventing the water droplet from coming into direct contact with the sensor element.

### Effect of the Invention

According to the present invention, a gas sensor can be obtained which can enhance both wetting resistance and responsiveness by using a single-wall protector.

### BRIEF DESCRIPTION OF THE DRAWINGS

FIG. 1 is a sectional view of a gas sensor according to an embodiment of a first mode of the present invention.

FIG. 2 is a plan view of a protector (gas introduction holes) as viewed from a forward end side toward a rear end side.

FIG. 3 is a partial enlarged view of the protector of FIG. 1 and its vicinity.

FIG. 4 is a sectional view of a gas sensor 1 according to an embodiment of a second mode of the present invention.

FIG. 5 is a sectional view showing an embodiment in which gas introduction holes differ in facing direction.

FIG. 6 is a sectional view showing the case where the interior of the protector can be seen when a gas introduction hole is viewed from a radially outer side.

### DESCRIPTION OF REFERENCE NUMERALS

Reference numerals used to identify various features in the drawings include the following.
1 gas sensor
11 metallic shell
11e rearward facing surface of the metallic shell
12a, 120a1, 120a2 forwardly facing surface of the metallic shell
12f, 120f forwardmost end of the forwardly facing surface
21 sensor element
22 detection section
25 element introduction hole
51, 151 protector
53, 153 gas discharge hole
56, 156 gas introduction hole
O axial line

### DETAILED DESCRIPTION OF THE PREFERRED EMBODIMENTS

The invention will next be described in greater detail with reference to the drawings. However, the present invention should not be construed as being limited thereto.

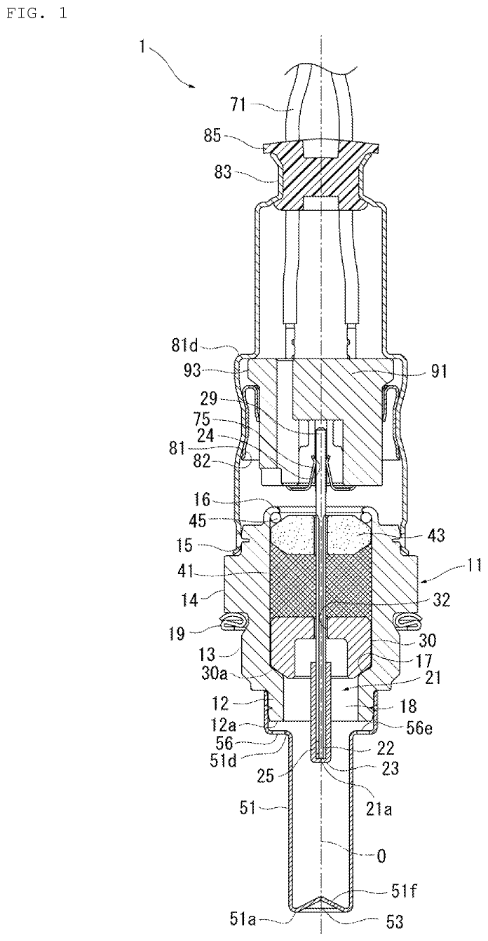

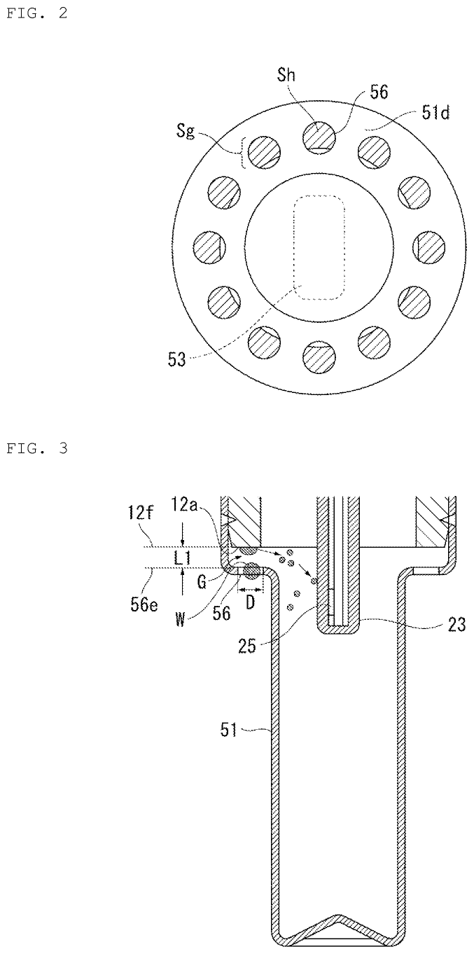

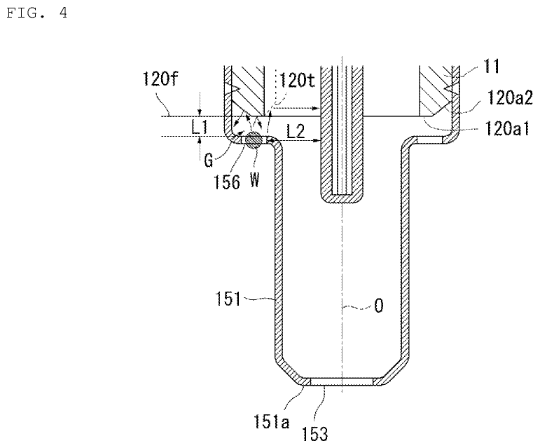

An embodiment of the first mode of the present invention will now be described in detail with reference to FIGS. 1 to 3. FIG. 1 is a sectional view of a gas sensor 1 according to the embodiment of the first mode of the present invention. FIG. 2 is a plan view of a protector 51 (gas introduction holes 56) as viewed from a forward end side toward a rear end side. FIG. 3 is a partial enlarged view of the protector of FIG. 1 and its vicinity.

In FIG. 1, the gas sensor (full-range air-fuel-ratio gas sensor) 1 includes a sensor element 21; a holder (ceramic holder) 30 having a through hole 32 which penetrates in the direction of an axial line O and into which the sensor element 21 is inserted; a metallic shell 11 which surrounds a circumference of the ceramic holder 30 in a radial direction; and a protector 51.

A forward end portion of the sensor element 21, where a detection section 22 is formed, projects forward from the ceramic holder 30 and the metallic shell 11. The sensor element 21, which is inserted into the through hole 32, is fixed inside the metallic shell 11, while gas tightness in a forward-rearward direction is secured. This is as a result of a seal material (talc in the present example) 41, which is disposed on the rear end surface side (the upper side in FIG. 1) of the ceramic holder 30, being compressed in the forward-rearward direction via a sleeve 43 formed of an insulating material and a ring washer 45.

Notably, a rear end portion 29 of the sensor element 21 projects rearward from the sleeve 43 and the metallic shell 11. Metallic terminal members 75 provided at tips of respective lead wires 71 extending to the outside through a seal member 85 are brought into pressure contact with respective electrode terminals 24 formed on the rear end portion 29, thereby being electrically connected to the respective electrode terminals 24. Also, the rear end portion 29 of the sensor element 21, which portion includes the electrode terminals 24, are covered by an outer casing 81. A further detailed description will be given below.

The sensor element 21 extends in the direction of the axial line O and has a strip-plate-like shape (plate-like shape). The sensor element 21 has a detection section 22 provided on a forward end portion (the lower side in FIG. 1) directed toward a measurement target. The detection section 22 is composed of detection electrodes, etc. (not shown) and detects a particular gas component contained in a gas to be detected. The sensor element 21 has a rectangular transverse cross section whose size is constant in the forward-rearward direction. The sensor element 21 is formed mainly of a ceramic material (solid electrolyte, etc.) such that the sensor element 21 has an elongated shape. This sensor element 21 itself is the same as a conventionally known sensor element. A pair of detection electrodes which constitute the detection section 22 are disposed on a forward end portion of the solid electrolyte (member), and the electrode terminals 24 which are connected to the detection electrodes and to which the lead wires 71 for taking out detection output are connected are formed on a rear end portion of the solid electrolyte (member) to be exposed to the outside.

In the present example, a heater (not shown) is provided in a forward end portion of a ceramic member laminated on the solid electrolyte (member) of the sensor element 21. Electrode terminals 24 to which lead wires 71 for applying a voltage to the heater are connected are formed on a rear end portion of the ceramic member to be exposed to the outside. Notably, although not illustrated, these electrode terminals 24 are formed to have a longitudinally elongated rectangular shape and are disposed on the rear end portion 29 of the sensor element 21, for example, in such a manner that three or two electrode terminals are juxtaposed laterally on each of wider surfaces (opposite surfaces) of the strip plate.

Notably, a porous protective layer 23 formed of alumina, spinel, or the like is provided to cover the detection section 22 of the sensor element 21. Also, the sensor element 21 has an element introduction hole 25 which communicates with the detection section 22 and through which the gas to be detected is introduced into the detection section 22. An unillustrated porous diffusion resistor layer is disposed in the element introduction hole 25.

The metallic shell 11 is tubular and has concentric portions which are juxtaposed in the forward-rearward direction and have different diameters. Specifically, the metallic shell 11 has a cylindrical annular portion (hereinafter also referred to as the cylindrical portion) 12 which is provided on the forward end side, which is small in diameter, and onto which a protector 51 (described below) is fixedly fitted. A screw 13 which is larger in diameter than the cylindrical portion 12 and is used for fixing to an exhaust pipe of an engine is provided on an outer circumferential surface on the rearward side (the upper side in the drawing) of the cylindrical portion 12. The metallic shell 11 has a polygonal tool engagement portion 14 provided on the rearward side of the screw 13. The tool engagement portion 14 is used to screw the screw 13, thereby attaching the sensor 1. Also, the metallic shell 11 has a cylindrical portion 15 provided on the rearward side of the tool engagement portion 14. The protecting tube (outer casing) 81, which covers a rear portion of the gas sensor 1 is fitted onto and welded to the cylindrical portion 15. The metallic shell 11 has a cylindrical portion for crimping 16 provided on the rearward side of the cylindrical portion 15. The cylindrical portion for crimping 16 has an outer diameter and a wall thickness smaller than those of the cylindrical portion 15.

Notably, in FIG. 1, the cylindrical portion for crimping 16 has an inwardly bent shape because the cylindrical portion for crimping 16 has been crimped. Notably, a gasket 19 is attached to a lower surface of the tool engagement portion 14. The gasket 19 establishes a seal when the screw 13 is fixed to an exhaust pipe.

Meanwhile, the metallic shell 11 has an internal hole 18 which penetrates the metallic shell 11 in the direction of the axial line O. The inner circumferential surface of the internal hole 18 has a step portion 17 tapered such that the step portion tapers radially inward from the rear end side toward the forward end side.

The holder (ceramic holder) 30, which is formed of an insulating ceramic material (for example, alumina) and is formed into an approximately short cylindrical shape, is disposed inside the metallic shell 11. The ceramic holder 30 has a forwardly facing surface 30a which is tapered down toward the forward end. As a result of the ceramic holder 30 being pressed by the seal material 41 from the rear end side, while a radially outer portion of the forwardly facing surface 30a is engaged with the step portion 17, the ceramic holder 30 is positioned in the metallic shell 11 and loosely fitted thereinto.

Meanwhile, the through hole 32 is provided at the center of the ceramic holder 30. The through hole 32 is a rectangular opening whose size is approximately the same as the transverse cross section of the sensor element 21 so that the sensor element 21 extends through the through hole 32 with substantially no clearance.

The sensor element 21 extends through the through hole 32 of the ceramic holder 30, and the forward end 21a of the sensor element 21 projects forward in relation to the ceramic holder 30 and the forward end 12a of the metallic shell 11.

Meanwhile, a forward end portion of the sensor element 21 is covered with a single-wall protector (protection cover) 51 having the shape of a bottomed cylinder. The rear end of the protector 51 is fitted onto and welded to the cylindrical portion 12 of the metallic shell 11. The protector 51 has a step portion 51d formed at a location near the rear end. The step portion 51d extends in the radial direction (direction perpendicular to the direction of the axial line O). The protector 51 has a smaller diameter on the forward end side of the step portion 51d.

Gas introduction holes 56 facing toward the rear end side are formed in the step portion 51d. As shown in FIG. 2, in the present example, a plurality (twelve) gas introduction holes 56 are provided at equal intervals in the circumferential direction of the step portion 51d. Notably, the expression “facing toward the rear end side” means that a perpendicular line of a plane 56e that passes through a portion of the circumferential edge of each gas introduction hole 56, which portion is located on the inner side of the protector 51, forms an angle in relation to the radial direction (is not parallel to the radial direction), and the portion of the circumferential edge of each gas introduction hole 56, which portion is located on the inner side of the protector 51, is located rearmost. In other words, the portion of the circumferential edge of each gas introduction hole 56, which portion is located on the inner side of the protector 51, is located on the rear end side in relation to a portion of the circumferential edge of the gas introduction hole 56, which portion is located on the outer side of the protector 51.

Meanwhile, a gas discharge hole 53 (one in the present example) is provided at the center of a bottom portion 51a of the protector 51. The gas discharge hole 53 is disposed on the forward end side in relation to the gas introduction hole 56. Because of the flow of the gas to be detected which flows through a mounting target (exhaust pipe, etc.) to which the gas sensor 1 is attached, gas within the protector 51 is sucked to the outside through the gas discharge hole 53, whereby a negative pressure is generated. Because of this negative pressure, the gas to be detected is introduced into the protector 51 through the gas introduction holes 56.

Notably, in the example of FIG. 1, a central portion of the bottom portion 51a of the protector 51 is allowed by two parallel slits to be raised toward the rear end side, thereby forming a cover 51f, and the gas discharge hole 53 is formed in the gap between the bottom portion 51a of the protector 51 and the cover 51f such that the gas discharge hole 53 extends in the radial direction. In this case, when the protector 51 is viewed from the forward end side in the direction of the axial line O, the gas discharge hole 53 can not be seen directly. Therefore, it is possible to restrain a droplet of condensed water or the like from invading into the protector 51 through the gas discharge hole 53.

Also, as shown in FIG. 1, due to the spring characteristics of the respective metallic terminal members 75, the respective metallic terminal members 75, which are provided at the tips of the respective lead wires 71 extending to the outside through the seal member 85, are brought into pressure contact with respective electrode terminals 24 formed on the rear end portion 29 of the sensor element 21, whereby the respective metallic terminal members 75 are electrically connected to the respective electrode terminals 24. In the gas sensor 1 of the present example, the respective metallic terminal members 75, including their pressure contact portions, are provided to face each other in respective accommodation spaces provided in an insulating separator 91 disposed in the outer casing 81. Notably, movements of the separator 91 in the radial direction and toward the forward end side are restricted by a holding member 82 fixedly held within the outer casing 81 by means of crimping. A forward end portion of the outer casing 81 is fitted onto and welded to the cylindrical portion 15 on the rear end side of the metallic shell 11, whereby a rear portion of the gas sensor 1 is gastightly covered.

Notably, the lead wires 71 extend to the outside through the seal member (for example, rubber) 85 disposed inside a rear end portion of the outer casing 81. This seal member 85 is compressed by reducing the diameter of this small diameter tubular portion 83 by means of crimping, whereby the gastightness of this portion is maintained.

The outer casing 81 has a step portion 81d which is formed at a position slightly shifted toward the rear end side from the center in the direction of the axial line O in such manner that the outer casing 81 has a larger diameter on the forward end side of the step portion 81d. The inner surface of this step portion 81d supports the separator 91 while pressing forward the rear end of the separator 91. Meanwhile, the separator 91 has a flange 93 formed on the outer circumference thereof, and the flange 93 is supported on the holding member 82 fixed to the inner side of the outer casing 81, whereby the separator 91 is held in position in the direction of the axial line O by the step portion 81d and the holding member 82.

Next, the characteristic portion of the first mode of the present invention will be described.

As shown in FIGS. 1 and 3, in the present embodiment, a gap G is provided between the forwardly facing surface 12a of the metallic shell 11 and the gas introduction holes 56, which face the forwardly facing surface 12a in the direction of the axial line O. Also, as shown in FIG. 2, when the gas introduction holes 56 are viewed toward the rear end side in the direction of the axial line O, the area Sh of portions of the forwardly facing surface 12a of the metallic shell 11, which portions can be seen through the gas introduction holes 56, is equal to or greater than ½ of the opening area Sg of the gas introduction holes 56. Notably, each of the areas Sh and Sg is a total area. For example, the area Sg is the sum total of the opening areas of the twelve gas introduction holes 56.

Notably, in the present embodiment, each of all the gas introduction holes 56 is formed such that an area Sh1 is also equal to or greater than ½ of an opening area Sg1. The areas Sh1 and Sg1 respectively represent the areas Sh and Sq individually determined for each gas introduction hole 56.

Furthermore, the entirety of the element introduction hole 25 is located on the forward end side in relation to the forwardmost end 12f of the forwardly facing surface 12a of the metallic shell 11, the distance L1 of the gap G (the distance between the forwardmost end 12f and the plane 56e) is smaller than the diameter D of the gas introduction holes 56.

In the case where the distance L1 is rendered smaller than the diameter D, when a water droplet W of condensed water or the like flows, together with the gas to be detected, from the gas introduction hole 56 toward the metallic shell 11, the water droplet W strikes the forwardly facing surface 12a of the metallic shell 11 without fail, whereby the water droplet W is broken to form fine water droplets. Therefore, even when the water droplets change their flow direction, flow toward the forward end side within the protector 51, and come into contact with the forward end portion of the sensor element 21, the sensor element 21 receives a reduced thermal shock as compared to the case where a water droplet W having a large diameter comes into contact with the sensor element 21. Therefore, the sensor element becomes less likely to break and wetting resistance can be enhanced.

Since the relation of Sh≥½×Sg is satisfied, one half or more of the opening area of the gas introduction holes 56 faces the forwardly facing surface 12a of the metallic shell 11. Therefore, the possibility of the water droplet W flowing from the gas introduction holes 56 toward the metallic shell 11 to strike the forwardly facing surface 12a increases, whereby such configuration can more reliably cause the water droplet W to strike the forwardly facing surface 12a, to thereby form fine water droplets.

Further, since the protector 51 is single walled, the gas to be detected flows easily into the protector as compared with a double-wall protector, and responsiveness is improved.

Also, although it is sufficient that the distance L1 is greater than 0, when the distance L1 is greater than 0.5 mm, the gas can be easily introduced into the protector 51. Therefore, the distance L1 is preferably greater than 0.5 mm.

Notably, the reason why the distance L1 is rendered smaller than the diameter D is as follows. The maximum diameter of water droplets W introduced into the protector 51 through the gas introduction holes 56 is D. Therefore, if the distance L1 is equal to or larger than the diameter D, when a water droplet W having the diameter D enters the gap G through the gas introduction holes 56, there is a possibility of the water droplet W moving within the protector 51 without striking the forwardly facing surface 12a and coming into contact with the sensor element 21 while maintaining its large size.

Also, the reason why the entirety of the element introduction hole 25 must be located on the forward end side in relation to the forwardmost end 12f is as follows. Namely, the gas to be detected introduced from the gas introduction holes 56 changes the flow direction at the position of the forwardmost end 12f of the forwardly facing surface 12a and flows toward the forward end side within the protector 51. Since the entirety of the element introduction hole 25 is located on the forward end side in relation to the forwardmost end 12f, it is possible to reliably bring the gas to be detected into contact with the detection section 22 of the sensor element 21 and detect the gas. Therefore, detection accuracy is increased.

In the case where at least a portion of the element introduction hole 25 is located on the forward end side in relation to the plane 56e of the gas introduction hole 56, it is possible to more reliably bring the gas to be detected into contact with the detection section 22 of the sensor element 21 and detect the gas. Therefore, detection accuracy is further increased.

Notably, in the case where a plurality of gas introduction holes 56 are provided as shown in FIG. 2, it is the best that the individual gas introduction holes 56 and the forwardmost end 12f of the forwardly facing surface 12a of the metallic shell 11, which the gas introduction holes 56 face individually, satisfy the relationship of L1<D for each of the gas introduction holes 56. However, in the case where at least one of the plurality of gas introduction holes 56 satisfies the relationship of L1<D, it is possible to reduce the amount of water droplets which reach the sensor element 21 while maintaining their large size.

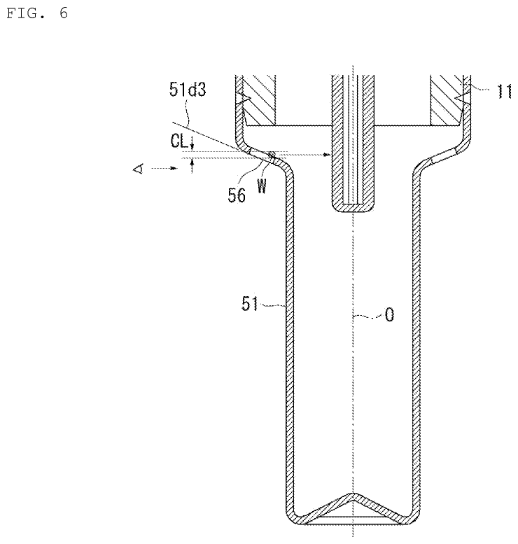

Also, in consideration of the case where the plane 56e is inclined as shown in, for example, FIGS. 5 and 6, described below, the distance L1 determined for each gas introduction hole 56 is the shortest distance between the forwardmost end 12f and a portion of the plane 56e at the circumferential edge of the gas introduction hole 56.

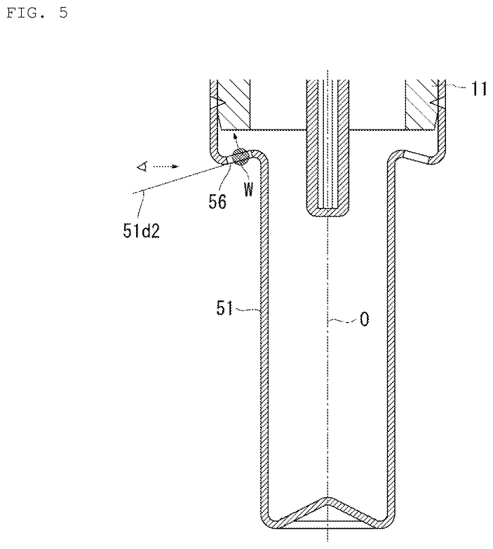

Next, an embodiment of the second mode of the present invention will be described in detail with reference to FIG. 4. FIG. 4 is a partial enlarged view of a protector 151 and its vicinity in a gas sensor according to the embodiment of the second mode of the present invention.

Notably, since the gas sensor according to the embodiment of the second mode is identical in structure with the gas sensor according to the embodiment of the first mode except for forwardly facing surfaces 120a1 and 120a2 of the metallic shell 11 and the protector 151, a description and illustration of identical portions will be omitted.

As shown in FIG. 4, in the present embodiment as well, a gap G in the direction of the axial line O is present between the gas introduction holes 156 and the forwardly facing surfaces 120a1 and 120a2 of the metallic shell 11. Also, although not illustrated, when the gas introduction holes 156 are viewed toward the rear end side in the direction of the axial line O, the area Sh of portions of the forwardly facing surfaces 120a1 and 120a2 of the metallic shell 11, which portions can be seen through the gas introduction holes 156, is equal to or greater than ½ of the opening area Sg of the gas introduction holes 156.

Furthermore, the entirety of the element introduction hole 25 is located on the forward end side in relation to the forwardmost end 120f of the forwardly facing surfaces 120a1 and 120a2 of the metallic shell 11, and the distance L1 of the gap G is smaller than the distance L2 in the radial direction between the sensor element 21 and the gas introduction holes 156.

In this way, the distance L1 is rendered smaller than the distance L2. Therefore, when a water droplet W flows, together with the gas to be detected, from the gas introduction holes 156 toward the metallic shell 11, the water droplet W strikes the forwardly facing surfaces 120a1 and 120a2 of the metallic shell 11 without fail, whereby the water droplet W is broken to form fine water droplets. Therefore, also in the second mode, the sensor element 21 receives a reduced thermal shock as compared to the case where a water droplet W having a large diameter comes into contact with the sensor element. Therefore, the sensor element becomes less likely to be broken and wetting resistance can be enhanced.

Also, although it is sufficient that the distance L1 is greater than 0, when the distance L1 is greater than 0.5 mm, the gas can be easily introduced into the protector 51. Therefore, the distance L1 is preferably greater than 0.5 mm.

The second mode differs from the first mode in that the relation of L1<L2 is prescribed. The reason why the relation of L1<L2 is prescribed is as follows. That is, the diameter (the maximum diameter) of a largest water droplet W which enters the gap G through the gas introduction holes 156 and then moves within the protector 151 without striking the forwardly facing surface 120a1, 120a2 is equal to the distance L1. In view of the above, the distance L1 is rendered smaller than the distance L2. In this case, even when a water droplet W has a diameter equal to the distance L1 (the maximum diameter), the water droplet W flows while keeping a distance from the sensor element 21. Therefore, the possibility of the water droplet W coming into contact with the sensor element 21 is decreased.

Notably, in the example of FIG. 4, the forwardly facing surface of the metallic shell 11 constitutes the horizontal surface 120a1 parallel to the radial direction, and the taper surface 120a2 which is connected to the radially outer side of the horizontal surface 120a1, tapers toward the forward end side, and faces the radially outer side.

In the case where, as described above, the configuration in which the forwardly facing surface of metallic shell 11 does not have a “taper surface which tapers toward the forward end side and faces the radially inner side” is employed, the water droplet W having flowed through the gas introduction holes 156 and striking the horizontal surface 120a1 or the taper surface 120a2 flows downward (the horizontal surface 120a1) or flows toward the radially outer side (the taper surface 120a2). Namely, the water droplet W bounces in directions which are not directions toward the radially inner side. Therefore, the water droplet W becomes less likely to come into contact with the sensor element 21.

In contrast, in the case where, as indicated by a broken line in FIG. 4, the forwardly facing surface of the metallic shell 11, which faces the gas introduction holes 156, has a “taper surface 120t which tapers toward the forward end side and faces toward the radially inner side,” the water droplet W striking this taper surface 120t bounces toward the radially inner side and approaches the sensor element 21. Therefore, as compared with the case where the forwardly facing surface of the metallic shell 11 has a horizontal surface or a taper surface facing the radially outer side, the water droplet W becomes more likely to come into contact with the sensor element 21, whereby the sensor element 21 is wetted with water. As a result, wetting resistance may decrease.

Therefore, the taper surface desirably does not face the radially inner side, but rather the horizontal surface and the taper surface facing the radially outer side face the gas introduction holes 156. It is also desired that one half or more of the opening area of each gas introduction hole 156 faces the horizontal surface or the taper surface facing the radially outer side. However, the present invention does not exclude the case where the forwardly facing surface of the metallic shell 11 has an inwardly facing taper surface, so long as wetting resistance is not decreased.

Notably, in the example of FIG. 4, of the horizontal surface 120a1 and the taper surface 120a2, the horizontal surface 120a1 corresponds to the “forwardmost end 12f of the forwardly facing surface of the metallic shell 11.”

Also, in the example of FIG. 4, a gas discharge hole 153 is directly formed at the center of a bottom portion (forward end portion) 151a of the protector 151.

Notably, in the case where a plurality of gas introduction holes 156 are provided in the second mode, the individual gas introduction holes 156, the forwardmost end 12f of the forwardly facing surface 12a of the metallic shell 11, which the gas introduction holes 56 face individually, and the gas sensor 21 must satisfy the relationship of L1<L2 for each of the gas introduction holes 156.

Also, the distance L2 determined for each gas introduction hole 156 is the shortest distance between (the circumferential edge of) the gas introduction hole 156 and the sensor element 21, which that gas introduction holes 56 faces.

The structure and configuration of the gas sensor of the present invention may be changed freely without departing from the scope of the present invention.

For example, in the above-described embodiments, the step portion 51d of the protector 51 is formed to extend in the radial direction (parallel to the radial direction), and the perpendicular line of the plane passing through a portion of the edge of each gas introduction hole 56 provided in the step portion 51d, which portion is located on the inner side of the protector 51, is perpendicular to the radial direction. However, the step portion of the protector 51 may be formed to have a non 90 degree angle with respect to the radial direction.

Specifically, as shown in FIG. 5, a step portion 51d2 of the protector 51 (a portion of the step portion 51d2 on the outer surface side of the protector 51) may be tapered such that the step portion 51d2 descends (to the forward end side) toward the radially outer side. In this case, the perpendicular line of the plane passing through a portion of the edge of each gas introduction hole 56 provided in the step portion 51d2, which portion is located on the inner side of the protector 51, (this perpendicular line represents the flow direction of the water droplet W entering the protector 51 through the gas introduction hole 56) inclines outward in the radial direction toward the rear end. Namely, since the water droplet W entering the protector 51 through the gas introduction holes 56 flows in a direction away from the sensor element 21, the possibility that the water droplet W comes into contact with the sensor element 21 is decreased.

Notably, in the example of FIG. 5 and the above-described examples of FIGS. 3 and 4, when the gas introduction holes 56 (156) are viewed from the outer side in the radial direction, the interior of the protector 51 (151) cannot be seen. When such a configuration is employed, it is possible to prevent the water droplet W from entering the protector in the radial direction, thereby preventing the water droplet W from coming into direct contact with the sensor element 21.

Here, we consider the case where, as shown in FIG. 6, a step portion 51d3 of the protector 51 is tapered such that the step portion 51d3 descends (to the forward end side) toward the radially inner side and, when each gas introduction hole 56 is viewed from the outer side in the radial direction, the interior of the protector 51 can be seen. In this case, when each gas introduction hole 56 is viewed from the outer side in the radial direction, a clearance CL can be seen. There is a possibility that the water droplet W flows into the protector through the clearance CL and comes into direct contact with the sensor element 21, whereby the wetting resistance is lowered.

Accordingly, it is preferred that, when each gas introduction hole is viewed from the outer side in the radial direction, the interior of the protector cannot be seen. However, a configuration may be employed in which, as shown in FIG. 6, the step portion 51d3 of the protector 51 is tapered such that the step portion 51d3 descends (to the forward end side) toward the radially inner side, but the interior of the protector 51 cannot be seen when the gas introduction holes 56 are viewed from the outside in the radial direction. The configuration in which the interior of the protector 51 cannot be seen can be realized, for example, by adjusting the angle of the step portion 51d3, the thickness of the protector 51, or the diameter of the gas introduction holes 56.

Also, the sensor element is not limited to those for measuring the concentration of oxygen and may be a sensor for measuring the concentration of nitrogen oxide (NOx), hydrocarbon (HC), etc.

A tubular sensor element may be used.

No limitation is imposed on the shapes and numbers of the gas introduction holes and the gas discharge hole. For example, the gas introduction holes and/or the gas discharge hole may have an elliptical shape. The shape of the forwardly facing surface of the metallic shell is not limited to the above-described shape.

The invention has been described in detail with reference to the above embodiments. However, the invention should not be construed as being limited thereto. It should further be apparent to those skilled in the art that various changes in form and detail of the invention as shown and described above may be made. It is intended that such changes be included within the spirit and scope of the claims appended hereto.

1. A gas sensor comprising:
a sensor element which extends in an axial direction and has a detection section formed in a forward end portion and detecting a gas to be detected through an element introduction hole;
a tubular metallic shell which surrounds a circumference of the sensor element in a radial direction and holds the sensor element; and
a single-wall tubular protector fixed to a circumference of a forward end portion of the metallic shell and surrounding the forward end portion of the sensor element, wherein
the protector has a gas introduction hole facing a rear end side and a gas discharge hole disposed on a forward end side in relation to the gas introduction hole;
a gap G in the axial direction is present between the gas introduction hole and a forwardly facing surface of the metallic shell;
when the gas introduction hole is viewed toward the rear end side in the axial direction, an area Sh of a portion of the forwardly facing surface of the metallic shell, which portion can be seen through the gas introduction hole, is equal to or greater than ½ of an opening area Sg of the gas introduction hole;
the entirety of the element introduction hole is located on the forward end side in relation to a forwardmost end of the forwardly facing surface of the metallic shell; and
a distance L1 of the gap G is smaller than a diameter D of the gas introduction hole.

2. A gas sensor comprising:
a sensor element which extends in an axial direction and has a detection section formed in a forward end portion and detecting a gas to be detected through an element introduction hole;
a tubular metallic shell which surrounds a circumference of the sensor element in a radial direction and holds the sensor element; and
a single-wall tubular protector fixed to a circumference of a forward end portion of the metallic shell and surrounding the forward end portion of the sensor element, wherein
the protector has a gas introduction hole facing a rear end side and a gas discharge hole disposed on a forward end side in relation to the gas introduction hole;
a gap G in the axial direction is present between the gas introduction hole and a forwardly facing surface of the metallic shell;
when the gas introduction hole is viewed toward the rear end side in the axial direction, an area Sh of a portion of the forwardly facing surface of the metallic shell, which portion can be seen through the gas introduction hole, is equal to or greater than ½ of an opening area Sg of the gas introduction hole;
the entirety of the element introduction hole is located on the forward end side in relation to a forwardmost end of the forwardly facing surface of the metallic shell; and
a distance L1 of the gap G is smaller than a distance L2 between the sensor element and the gas introduction hole in the radial direction.

3. The gas sensor as claimed in claim 1, wherein the forwardly facing surface of the metallic shell constitutes a horizontal surface parallel to the radial direction and/or a taper surface which tapers toward the forward end side and faces a radially outer side.

4. The gas sensor as claimed in claim 1, wherein, when the gas introduction hole is viewed from an outer side in the radial direction, the interior of the protector cannot be seen.

5. The gas sensor as claimed in claim 2, wherein the forwardly facing surface of the metallic shell constitutes a horizontal surface parallel to the radial direction and/or a taper surface which tapers toward the forward end side and faces a radially outer side.

6. The gas sensor as claimed in claim 2, wherein, when the gas introduction hole is viewed from an outer side in the radial direction, the interior of the protector cannot be seen.
